(19) United States
(12) Patent Application Publication
(10) Pub. No.: US 2007/0148674 A1
(43) Pub. Date: Jun. 28, 2007

(54) OPTICAL FINGERPRINTING OF NUCLEIC ACID SEQUENCES

(75) Inventors: Mark E. BERRES, Madison, WI (US); David A. Frisch, Fitchburg, WI (US); Fredrick R. Blattner, Madison, WI (US)

Correspondence Address: GODFREY & KAHN, S.C., 780 N. WATER STREET, MILWAUKEE, WI, 53202, US

(73) Assignee: Wisconsin Alumni Research Foundation (WARF)

(21) Appl. No.: 11/615,695

(22) Filed: Dec. 22, 2006

Related U.S. Application Data
(60) Provisional application No. 60/753,564, filed on Dec. 23, 2005.

(51) Int. Cl. C12Q 1/68; C12M 3/00

(52) U.S. Cl. 435/6; 435/287.2; 977/924

(57) ABSTRACT

The present invention provides methods and apparatus for the optical fingerprinting of nucleic acid molecules. In certain embodiments, the invention provides methods for labeling nucleic acid molecules using-site-specific nucleic acid binding partners that bind to nucleic acids without cleaving the molecule. The nucleic acid binding partners can be labeled directly with a fluorophore, such as a quantum dot (QD), or indirectly. Examples of suitable binding partners include cut-deficient restriction endonucleases (cdREs), transcription factors, the binding domains of nucleic acid polymerases, antibodies and the like. The methods disclosed make the assembly of fingerprint contigs easier and allows for digitizing the results so as to provide for easier computer manipulation and assembly. The invention also includes a microarray, the microarray designed to provide for easy deposition and high-throughput fingerprinting. The invention also provides for methods of using the microarrays. The invention also provides kits for the use of the invention.

### CROSS REFERENCE TO RELATED APPLICATIONS

This application claims the benefit of U.S. Provisional Application 60/753,564 filed on Dec. 23, 2005, incorporated herein by reference in its entirety for all purposes.

### GOVERNMENT SUPPORT

Development of this invention was supported in part by grants from the National Institutes of Health Grant HG002530. The Government of the United States of America may have certain rights in this invention.

### FIELD OF THE INVENTION

This invention is generally directed to methods of fingerprinting nucleic acids. More particularly, this invention is directed to methods of optically fingerprinting nucleic acid molecules using site-specific recognition molecules.

### BACKGROUND OF THE INVENTION

The technological explosion accompanying the growth in molecular biology has made possible the large-scale sequencing of complete genomes. However, such large-scale sequencing is limited by the comparative slowness of preparing individual genomes or genome libraries for large-scale nucleotide sequencing. Currently, libraries of genomic digests are made and the inserts are sequenced and then aligned to identify contiguous regions of the genome. Two types of vectors, the yeast artificial chromosome (YAC) and the bacterial artificial chromosome (BAC), are currently used for the construction of such libraries.

Both the YAC and the BAC vector based libraries have positive and negative aspects to their use. For example, the YAC vector can accommodate inserts of up to 4 megabases (Mb) while BAC vectors can only accommodate inserts of up to 300 kb, a limit imposed by decreased transfection efficiency. For sequencing purposes, YAC vectors are comparatively unstable and prone to chimerism of the insert. BAC libraries, however, are readily compatible with existing automated DNA purification and sequencing technologies. Therefore, except for the smaller size of the insert, BAC libraries are generally preferred for the cloning and sequencing of nucleic acids.

As technology improves, large-scale, high-throughput nucleotide sequencing will require rapid construction of high-fidelity vector based libraries. Next, the physical mapping of the library inserts will be necessary. Insert mapping identifies overlapping inserts (contigs) from different clones and provides a necessary and efficient means to organize, construct and verify the sequencing effort.

The most common practice used in mapping vector libraries is to identify overlapping clones with the DNA fingerprints generated by restriction endonuclease digestion. Two methods of restriction fingerprinting are commonly used to map BAC inserts. One identifies overlapping patterns of a small subset of digested fragments visualized in a polyacrylamide gel. The other relies on determining overlap from possibly dozens of larger fragments derived from the entire clone and separated in agarose gels. In both methods, individual fragments are assembled into a fingerprint contig, a set of overlapping segments of DNA, based on estimated fragment size and vector origin, providing a physical map of the entire BAC library. These approaches require substantial numbers of fragment-pair comparisons and require computer software to detect potential overlaps, calculate overlap probabilities and assemble the contigs.

Although high-quality maps can be created with these methods, they share significant technical limitations that reduce both information content and throughput. First, each mapping effort is restricted to a single restriction endonuclease. The specific endonuclease choice is conditioned on the number of fragments it generates. Too many or too few fragments create problems with resolution sensitivity and/or information content. Second, fingerprints obtained in this manner are unordered. Ordered restriction maps enhance reliable contig assembly and aides the alignment of nucleotide sequence information. Third, restriction fingerprinting has difficulty identifying small overlaps; minimally overlapping clones are most valuable in terms of gap closure and reducing overall sequencing redundancy. Fourth, if additional maps are desired, the entire fingerprinting procedure must be repeated with a different restriction endonuclease. This is rarely done in practice, as each map is independent with respect to its contig assembly. Merging different maps entails significant additional effort requiring either fragment hybridization or end sequencing. Even despite these technical issues, no matter which method is implemented, substantial human involvement is required and remains the rate-limiting step in fingerprint contig assembly.

Recently, a new method has been developed that generates contigs of BAC clones based on multiplexed fluorescence labeling (Ding et al., Genomics (2001) June 1; 74(2):142-54; Ding et al., Genomics (1999) Mar. 15; 56(3)237-46). This procedure uses automated sequencers to detect uniquely labeled fragments digested with paired restriction endonucleases for each BAC clone. This system improved the accuracy of fragment size calling and decreased the amount of human involvement by eliminating gel-based fragment separation, tracking and scoring. The ability to detect small overlaps was also possible with this technique using three pairs of restriction endonucleases.

Although higher throughput can be achieved with fluorescent fingerprinting, as described by Ding et al., numerous disadvantages remain. Unlike traditional agarose gel fingerprinting, the ability to determine the size of the BAC insert is no longer possible. Moreover, despite the capability to multiplex different restriction endonucleases per BAC clone, each map still remains independent with respect to its contig assembly. Further, the presence of chromosomal DNA in the preparation often produces extraneous fragments, a sensitivity issue not apparent in agarose separations. Thus, extremely pure DNA is required for fluorescent fingerprinting, as erroneous chromosomal fragments can impair automated contig assembly. Nevertheless, these methods remain the standard by which large-scale BAC mapping is performed.

Existing DNA sequencing technology has advanced to the point where large sequencing centers can rapidly generate megabase amounts of sequence data. Development of newer technologies is expected to increase further sequence throughput and decrease the cost per base. These improvements will inevitably lead to additional projects aimed at genome-level sequencing. However, it is doubtful that the preparation of sequence-ready BAC libraries can keep pace with current sequencing technologies. Prior to the completion of the human genome project, it was estimated that to contig and partially sequence a 30-fold BAC library by end-sequencing and fingerprinting clones would require as many as 600,000 BAC clones to generate a sequence-ready contig assembly. Even given a generous rate of 1,000 fingerprints per day, this effort would still require almost two years for completion. Traditional fingerprinting methods cannot be expected to keep pace even with existing sequencing technologies. Therefore, in order to meet the increasing need for genome-level sequencing projects, new systems of vector mapping must be developed.

A technology that does not rely on cloned or amplified sequences for hybridization or gel electrophoresis is optical mapping (Lim, A., et al., “Shotgun Optical Maps of the Whole Escherichia coli O157:H7 Genome”, Genome Res. 11: 1584-1593, 2001). This procedure uses light microscopy to construct physical maps from large regions of DNA, such as BACs, YACs, or entire genomes. Large DNA molecules are immobilized under slight tension onto a glass surface and digested with a single restriction endonuclease. Positions where the endonuclease has cut appear as breaks in an otherwise contiguous stretch of DNA. Using a relationship between DNA length and the amount of intercalation dye, the size of each restriction fragment is determined by measuring its relative fluorescence intensity. Ordered restriction maps are derived from digital images of fully and partially digested molecules. Akin to traditional fingerprinting methods, optical mapping provides an enhanced ability to align and verify sequence contigs, identify errors in contig assembly, locate and size gaps, and identify the order and size of each fragment.

Although optical mapping is an improvement over traditional mapping methods, it still shares some of the same limitations inherent to DNA fingerprinting. Optical maps cannot be easily multiplexed with more than one restriction endonuclease. Different optical maps of the same molecule, made with different restriction enzymes still cannot be integrated with each other without additional labor-intensive procedures such as Southern blotting due to the lack of internal landmarks. Thus, the resolution of the optical map is dictated by the average restriction fragment size which itself is dependent on a particular endonuclease. Optical maps also cannot reliably report restriction fragments smaller than 1.5 Kb. As the resolution of the map increases, the number of fragment dropouts also increases. This phenomenon creates an intrinsic upper-limit to map resolution. Thus, any attempt to increase the resolution of the map also increases the number of fragment dropouts. This phenomenon may be exacerbated in repetitive genomic regions, a hallmark of most eukaryotic genomes. Perhaps the most serious issue intrinsic to conventional optical mapping is the rate of false positives (a cut at a non-specific site or spontaneous DNA breakage interpreted as a cut) and negatives (failure to cut at a specific site). Although there is no doubt that high-quality maps can be constructed with optical mapping, the coverage at any given site must be considerable (in the range of 80-100×) to accommodate these phenomena, necessitating substantial allocation of resources to data acquisition and computational analysis, thereby decreasing overall throughput and increasing total project costs. Moreover, because of potential error rates, the use of optical mapping for investigations other than chromosomal mapping is implausible.

Currently, the best resolution achieved by optical maps is in the range of 7-15 Kb. As currently employed, optical mapping also cannot easily process the thousands of BAC clones that comprise a typical vertebrate library. Therefore, in order to meet the increasing need for genome-level sequencing projects, new systems of genome mapping must be developed.

### SUMMARY OF THE INVENTION

The present invention provides methods for the optical fingerprinting of nucleic acid molecules. In certain embodiments, the invention provides methods labeling nucleic acid molecules using site-specific nucleic acid binding partners that bind to nucleic acids without cleaving the molecule. The nucleic acid binding partners can be labeled directly with a fluorophore, such as a quantum dot (QD), or indirectly by recombinantly adding a biotin moiety to the binding molecule and using an avidin conjugated fluorophore to bind to the biotin moiety using standard biotin-avidin chemistry. Examples of suitable binding molecules include cut-deficient restriction endonucleases (cdREs), transcription factors, the binding domains of nucleic acid polymerases, antibodies and the like. The methods disclosed make the assembly of fingerprint contigs easier and provides for assembling the results so as to allow easier computer manipulation. In addition, by using functional moieties as binding partners, the invention allows, not only for sequence fingerprinting, but also for functional fingerprinting. The invention also includes a microarray designed to provide for easy deposition and high-throughput fingerprinting of nucleic acid molecules having sequence-specific binding partners bound to them. The invention also provides for methods of using the microarrays described so as to provide high-throughput fingerprinting and contig assembly of the nucleic acid molecules deposited on the microarray surface. The invention also provides kits for the use of the invention.

In preferred embodiments, the invention comprises a method of mapping a site of known sequence on a polynucleotide having first and second ends, comprising the steps of contacting the polynucleotide with a sequence-specific binding partner without scission of the polynucleotide; and determining the position, relative to a first landmark of the sequence-specific binding partner to map the position of a site of known sequence on the polynucleotide. In some embodiments, the landmark is an end of the polynucleotide, a cos site or a NotI site. In some preferred embodiments, the method further comprises another landmark that is a sequence-specific binding partner. In some embodiments, the binding partner comprises a peptide or a peptide nucleic acid. In some preferred embodiments, the binding partner comprises the DNA binding domain selected from the group consisting of a cut-deficient restriction endonuclease, an RNA polymerase, a nucleic acid binding antibody, a zinc-finger protein, a leucine zipper protein, a repressor protein, a TATA box-binding protein and a transcription factor.

In those preferred embodiments where the binding partner is a cut-deficient restriction endonuclease, the cut deficient endonuclease is a mutein of an endonuclease having a residue-specific recognition sequence. Typically, such recognition sequences are from 4 to 8 or more nucleotides long. In other preferred embodiments, the binding partner includes the binding domain or a zinc-finger protein, a steroid receptor or other transcription factor. In other embodiments the binding partner includes the binding domain of a nucleic acid polymerase without a sigma factor.

In some preferred embodiments, the binding partner further is linked to a detectable label. In some embodiments, the binding partner is directly linked to the detectable label. In other preferred embodiments, the binding partner is indirectly linked to the detectable label. In some embodiments, the binding partner is linked to the detectable label by an avidin-biotin association. In other preferred embodiments the binding partner is linked to the detectable label by and antibody-antigen association. In particularly preferred embodiments, the detectable label is a fluorophore, a chromophore, a quantum dot or a radionuclide.

The invention also includes a microarray for use in fingerprinting nucleic acids comprising a nucleic acid deposition apparatus. In some preferred embodiments the nucleic acid deposition apparatus includes a first chamber and a second chamber and an elongation channel connecting the first chamber to the second chamber. The deposition apparatus also includes a derivatized substrate adhered to the elongation channel. In this embodiment, the first chamber comprises an input well and the second chamber provides an output well and the elongation channel provides a nucleic acid deposition surface. In some preferred embodiments, the first and second chamber are formed by a top blocking mask and wherein the elongation channel is formed from a bottom blocking mask. In some embodiments, the top and bottom blocking masks are formed from polydimethylsiloxane (PDMS) and sit on top of a derivatized glass surface, the glass surface derivatized with 3-aminopropyltriethoxysilane (APTES).

To facilitate high-throughput screening of the nucleic acid molecules deposited on the surface of the elongation channel multiple deposition apparatus are placed on a single derivatized glass surface. In some preferred embodiments, at least 5000 nucleic acid deposition apparatus are present on a derivatized surface. In other preferred embodiments, 10,000 nucleic acid deposition apparatus are present on a derivatized surface. In still other preferred embodiments between 5,000 and 10,000 deposition apparatus are placed on a derivatized surface.

The invention also includes a method of depositing nucleic acid on a surface so as to be amenable to contig assembly via high-throughput screening. In some preferred embodiments, the method comprises the use of a microarray having a deposition apparatus as described above. In this embodiment, a solution containing a nucleic acid mixture is place in the input well and wherein flow dynamics causes the solution to pass through the elongation channel to the output well and wherein nucleic acids present in the input well deposited on the derivatized surface of the elongation chamber. In these preferred embodiments, the nucleic acids deposited on the surface of the elongation channel can be fingerprinted using site-specific binding partners as described above thereby providing for high-throughput fingerprinting and contig assembly of nucleic acids.

The invention also comprises a kit for use in mapping sites of known sequence on a polynucleotide comprising at least one binding partner that site-specifically binds the polynucleotide without resulting in scission of the polynucleotide and a detectable label linked thereto. In some embodiments, when the invention comprises a kit, the binding partner is a detectable label. In other preferred embodiments, the binding partner is biotinylated. In some preferred embodiments, the kit further includes a detectable label conjugated to an avidin moiety. In particularly preferred embodiments one or more quantum dots are conjugated to the avidin moiety.

These and other features and advantages of various preferred embodiments of the methods according to this invention are described in, or are apparent from, the following detailed description of various exemplary embodiments of the methods according to this invention.

### BRIEF DESCRIPTION OF THE FIGURES

Various exemplary embodiments of the methods of this invention will be described in detail, with reference to the following figures, wherein:

FIG. 1 shows solutions containing fluorescent CdSe/ZnS core-shell quantum dots excited by long wavelength UV light and emitting orange light (605 nm) or green light (525 nm).

FIG. 2 shows the purification of cut-deficient BamHI endonuclease D94N. Lane 1: Low-MW protein standard; Lane 2: clarified cell lysate; Lanes 3-5: 20 mM imidazole washes; Lanes 6-8: 1 μl samples from the first, second and third 0.5 ml elution with 200 mM imidazole.

FIG. 3 is a schematic illustration of the crystal structure of Bam HI showing one embodiment of the invention (i.e. AVITAG incorporation for biotinylation and 6×HIS tag for purification).

FIGS. 4A-4G are schematic representations of one preferred method of optical fingerprinting according to this invention. FIG. 4A, undigested BAC clone; FIG. 4B, BAC clone I-Scel linearized; FIG. 4C, BAC clone gpNu1 and Not I labeling; FIG. 4D, linearized BAC clone with binding of one cut-deficient restriction endonuclease (cdRE); FIG. 4E, linearized BAC clone with multiple cdREs (fingerprint); FIG. 4F, fingerprint with multiple cdREs corresponding digitized fingerprint; FIG. 4G, contig assembly.

FIGS. 5A and 5B show the results of mobility shift assays with the D94N protein. FIG. 5A EtBr stained gel of BpmI digested pGEM3Z with unlabelled D94N. Lane 1, marker; lane 2, pGEM3Z; lane 3, pGEM3Z+D94N cell lysate; lane 4, pGEM3Z+purified 6×His-D94N. FIG. 5B QD-conjugated D94N. lane 1, marker; lane 2, pGEM3Z; lane 3, pGEM3Z+QD labeled 6×His-D94N.

FIG. 6 illustrates the purification of biotinylated D94N with monomeric avidin. Lane 1: MW protein std; lane 2: column wash; lane 3: first few drops of eluate after column was loaded with D94N purified by Ni-NTA column; lane 4: first column wash; lane 5: final column wash; lanes 6-10: 2 ml elution fractions of biotinylated D94N.

FIG. 7 shows the results of a gel shift assay using increasing amounts of cut deficient Bam HI endonuclease D94N containing a biotinylation tag. Lane 1: low-MW DNA std; lane 2: 160 bp fragment containing a Bam HI site; lane 3: 160 bp fragment digested with BamHI; lanes 4-7: 160 bp fragment+100, 200, 300, and 400 pmoles D94N; lanes 8-11: 232 bp fragment with no BamHI site+100, 200, 300, and 400 pmoles D94N.

FIG. 8 is a visualization of QD-labeled D94N protein using a Leica TCS SP2 AOBS confocal microscope with 405 nm excitation.

FIG. 9 is a confocal image of QD-labeled streptavidin bound to biotinylated D94N.

FIG. 10, is a visualization of YOYO-1 stained λ DNA polynucleotide molecules using a Leica TCS SP2 AOBS confocal microscope with 405 nm excitation.

FIGS. 11A-11C are fluorescence images of QD-labeled streptavidin demarcating BamHI sites on a λ DNA polynucleotide. YOYO-1 intercalated DNA stains green and unbound QD-labeled streptavidin appears red. QD specifically bound to DNA via the D94N cut deficient enzyme are yellow. FIG. 11A shows QD white arrow, 201 bound to λ DNA 101 at position 22,346 (calculated=22,078). FIG. 11B shows QD bound to λ DNA 103 at positions 41,732 (calculated=42,464) white arrow 301 and 34,499 (calculated=33,906) white arrow 303. The gray arrow, 307, indicates a closely adjacent QD that is not bound. FIG. 11C shows: QDs bound to λ DNA 105 at positions 5,505 (calculated=5,905) white arrow 401 and 22,346 (calculated=23,940) white arrow 403. An incomplete molecule 107 is also shown (the broken portion overlaps with the curved strand 105 at gray arrow, 405). The calculated position, 5,453, corresponds to position 5,505 shown at white arrow 407. FIG. 11B demonstrates progress in reducing background binding of QD over FIG. 11C.

FIGS. 12A-12D shows embodiments of DNA elongation chambers. A derivatized microscope slide is overlaid with a microfabricated plastic layer containing two wells separated by a channel in which the fluid contacts the glass. DNA is added to the wells from above, from which it flows through the channel. The overall length of the unit in is 150 μm (FIG. 12A), 200 μm (FIG. 12B), 200 μm (FIG. 12C), and 200 μm (FIG. 12D).

FIGS. 13A and 13B are images of polyacrylamide gels illustrating the “gel-shift” that results from binding of BamHI to ScaI cut pGEM3Z. FIG. 13 A: Lane 0: Molecular weight standard; Lane 1: ScaI cut pGEM3Z; Lane 2: D94N-11+pGEM3Z (gel shift with cdRE D94N-11 with 6×HIS tag only); Lane 3: D94N-32+pGEM3Z (gel shift with cdRE D94N-32 with 6×HIS tag and AVITAG; Note: mixture of biotinylated/un-biotinylated forms); Lane 4: As lane 2 but with active BamHI added; Lane 5: As lane 3 but with active BamHI added; Lane 6: As lane 1 but with active BamHI added (positive cut control of single BamHI site in pGEM3Z); Lane 7: Replicate of lane 4; Lane 8: Replicate of lane 5. FIG. 13B: Lane 0: Molecular weight standard; Lane 1: ScaI cut pGEM3Z; Lane 2: purified biotinylated D94N-32 (b-D94N-32) Note: no longer a mixture of biotinylated and un-biotinylated forms; Lane 3: replicate of lane 2; Lane 4: Lane 2+active BamHI; Lane 5: replicate of lane 4; Lane 6: As lane 1 but with active BamHI added (positive cut control of single BamHI site in pGEM3Z).

### DETAILED DESCRIPTION OF THE EXEMPLARY EMBODIMENTS

Before the present invention is described, it is understood that this invention is not limited to the particular methodology, protocols, nucleic acid sequences, and reagents described, as these may vary. It is also to be understood that the terminology used herein is for the purpose of describing particular embodiments only, and is not intended to limit the scope of the present invention which will be limited only by the appended claims.

As used herein, the term “contacting” means that the components of the reaction used in the present invention are introduced to a substrate in a test tube, flask, tissue culture, chip, array, plate microplate, capillary, or the like and incubated at a temperature and time sufficient to permit a reaction to occur. The term “polynucleotide” means a polymer of mononucleotides or a nucleic acid. RNA and DNA are nucleic acids. The term “bind” means to combine or unite molecules by means of reactive groups, either in the molecules per se or in a chemical added for that purpose; frequently used in relation to chemical bonds that may be fairly easily broken (i.e., noncovalent), as in the binding of a toxin with antitoxin, or a heavy metal with a chelating agent, etc. The term “binding partner” means a molecule that binds to another molecule. The term “mutein,” as used herein, means a protein that arises as a result of a mutation.

As used herein, the term “vector” refers to a nucleic acid molecule capable of transporting another nucleic acid to which it has been linked. One type of vector is a “plasmid” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other types of vectors are yeast artificial chromosomes (short YAC) and bacterial artificial chromosomes (BAC). A YAC is a vector used to clone large nucleic acid fragments (up to 400 kb). It is an artificially constructed chromosome and contains the telomeric, centromeric, and replication origin sequences needed for replication in yeast host cells. A BAC is a vector used to clone nucleic acid fragments (up to 150 kb) having a telomere, centromere and repliation sequences needed for replication in a bacterial host cell. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. As used herein a “library” refers to an unordered collection of clones (i.e., cloned DNA, cDNA or RNA from a particular organism), whose relationship to each other can be established by physical mapping. The nucleotide sequences of interest are preserved as inserts to a plasmid. A cDNA library represents all of the mRNA present in a particular tissue, while a genomic library represents all of the DNA sequences found in the genome, broken into fragments of a manageable size. The nucleic acid inserts contained within the library vectors are polynucleotides.

As used herein, a “detectable label” has the ordinary meaning in the art and refers to an atom (e.g., radionuclide), molecule (e.g., fluorescein, chromophore, quantum dot), or complex, that is or can be used to detect (e.g., due to a physical or chemical property), to indicate the presence of a molecule, or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrophoretic, electrical, optical or chemical means. A tag is a detectable label.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

It must be noted that as used herein and in the appended claims, the singular forms “a”, “an”, and “the” include plural reference unless the context clearly dictates otherwise. Thus, for example, reference to “a cell” or “a label” includes a plurality of such cells or labels. Reference to the “vector” is a reference to one or more vectors and equivalents thereof known to those skilled in the art, and so forth.

Characteristics of Quantum Dots

Advances in nonisotopic detection methods have significantly influenced many areas of research ranging from fundamental molecular biology to bioimaging. Most optical methods require fluorescent probes to detect and monitor microscopic interactions. Examples of these labels include many well-known organic fluorophores and naturally occurring chromophores such as the green or red fluorescent protein and phycoerytherin. Despite their obvious success in applied research, organic fluorophores exhibit many undesirable characteristics including relatively weak spectral emissions, short fluorescence half-life, narrow excitation and broad emission spectra. Luminescent semiconductor nanocrystals (quantum dots; QDs) provide a much-improved alternative to traditional molecular reporters. Quantum dots can be orders of magnitude brighter and completely resistant to fading. Dependent only on their size, typically 3-10 nm (similar to small proteins), QDs also exhibit narrow (ca.≦40 nm) and symmetrical emission spectra that are excitable over a broad range of wavelengths, particularly in the UV spectrum. Importantly, a single excitation source can simultaneously excite QDs of distinguishable emission spectra. These optical properties provide the essential features needed for optical fingerprinting to easily visualize and multiplex cdREs labeled with differently colored QDs.

Synthesis of Core Quantum Dots

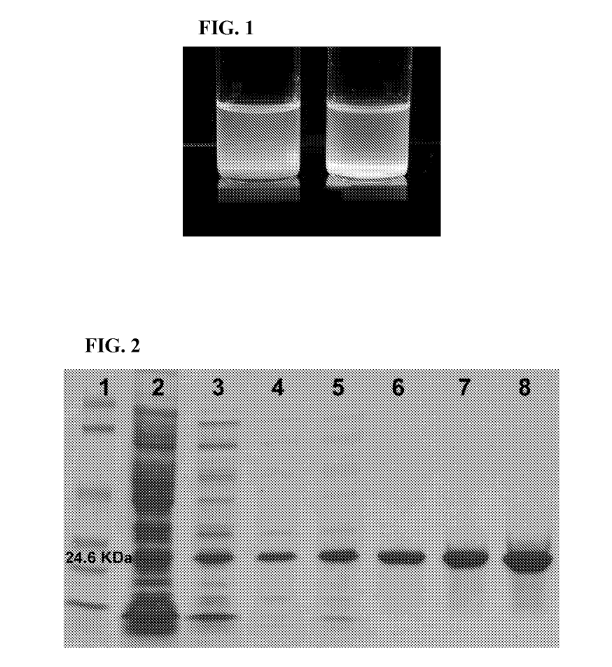

It was first shown that pyrolysis of metal-organic precursors could yield colloidal preparations of cadmium selenide (CdSe) core QDs with a coefficient of variance (CV) of the size distribution of approximately 9 percent. This basic methodology is used to produce high-quality QDs. This original approach has been modified to include an additional coordinating solvent, hexadecylamine (HDA). The inclusion of HDA provides much better control over the rate of crystal growth and further contributed to a reduction in the distribution of QD sizes (to approximately 5 percent). The inventors have produced numerous QD preparations exhibiting emission wavelengths ranging from 525 nm (green) to 655 nm (reddish-orange) as shown in FIG. 1. While it is possible to produce quantum dots in the laboratory, they can also be purchased commercially. Such commercially prepared QDs are available in multiple emission colors from, for example, Quantum Dot Corporation (Hayward, Calif.) and Evident Technologies (Troy, N.Y.). In addition, such commercially available QDs can be obtained pre-conjugated to antibody domains for immunohistochemical analysis or avidin, for ready use using avidin/biotin chemistry.

Purification of Core Quantum Dots

To further increase the optical purity (i.e. minimize size disparity) of bulk QDs, QDs were incrementally precipitated with methanol, which causes the larger QDs to precipitate from solution where they can be collected by simple centrifugation. Repeated precipitations provide populations of QDs of decreasing size. This method provides preparations of QDs that may vary in size by as little as 10%.

Synthesis of Core/Shell Quantum Dots

QD cores prepared using organometallic methods exhibit near perfect crystal structures and narrow size distributions, but the fluorescence quantum yield (QY) remains low (ca. 10-12 percent). The addition of a surface-capping layer (shell) such as ZnS or CdS dramatically increases the QY of CdSe QDs to approximately 50-60 percent. For CdSe cores with ZnS shells, approximately 1.6 monolayers of ZnS produce a QY of 66 percent. Here, a monolayer of ZnS is defined to be 0.31 nm thick. Shell thicknesses beyond 1.6 monolayers decrease the QY to approximately 50 percent.

Quantum Dot Surface Modification

Core-shell QDs are extremely hydrophobic and are rapidly destroyed when in contact with aqueous media. Protecting QDs against oxidation, while also providing reactive surface sites to allow bioconjugation, has been challenging. Most work has focused on adsorption of other surface ligands including dihydrolipoic acid, dithiothreitol, engineered proteins, and phospholipid micelles (PLMs). The inventors have experimented extensively with PLMs and silane shells to both solubilize QDs and provide bioconjugation sites. Results indicate that the PLM coated QDs are highly stable across a pH range of 4.5 to 9.0 in buffers lacking phosphate and detergent components. These extremes of pH are required for the condensation chemistry used for covalent modifications (below). Preparation of QD-containing PLMs requires only 15 minutes and does not need additional costly chemicals or equipment. Addition of reactive groups (e.g. amino, carboxy, sulfhydryl) only requires exchange of specific molecules containing the desired moiety. However, during the course of investigations reported herein, it has been found that the PLMs are stable only in buffers lacking phosphate and detergent components. Unfortunately, the use of detergents improves DNA elongation by reducing the surface tension of water when mounting DNA on derivatized glass slides. Despite a more involved synthesis requiring about three days, silanized QDs exhibit remarkable stability in detergent-containing solutions and in all buffer systems we require. Thus, the silane-shell solubilization method has been adopted for use in optical fingerprinting technology.

DNA Binding Molecules

There are a variety of nucleic acid binding molecules that are present in cells for various purposes. For example, as previously mentioned the restriction/methylase system is thought to act defensively by breaking down foreign DNA while protecting native DNA. In addition, gene regulation requires functional molecules, such as transcription factors, that bind site-specifically to nucleic acid sequences and act to repress or activate gene sequences. For example, gene promoters generally have response elements that bind cognate factors. These response elements include the TATA box, the CAAT box, the GC box, the octamer, and the activating transcription factor element. Each element binds its cognate ligand, the Tata binding protein, CaaT binding factor/necrosis factor 1, stimulator protein 1, Oct-1/Oct-2 and activating transcription factor (ATF) respectively. A host of other site-specific DNA binding partners have also been identified. These include RNA polymerase, the steroid hormone receptors such as the vitamin D receptor, the retinoic acid receptor and the retinoic X receptor as well as, stimulatory proteins 1 and 3 (Sp1 and Sp3) nuclear factor Y (NF-Y), upstream stimulatory factor (USF) and sterol regulatory element binding protein-1 (SREBP-1) to name a few. Generally, such transcription factors are grouped into the helix-turn-helix, zinc-finger, leucine zipper, and helix-loop-helix protein families of transcription factors. Each binding partner binds its cognate element and may activate transcription or repress which may depend on the presence of other factors bound to their cognate site.

Restriction Endonucleases

Restriction endonucleases are components of bacterial restriction modification systems that are thought to effect the cleavage of foreign DNA and modification (methylation) of native DNA, protecting it from action of the endonucleases. Restriction endonucleases are generally grouped into four classes (I-IV) based on subunit composition and cofactor usage. By far the most used and the best studied are the type II endonucleases which are used in the bulk of molecular biological work.

While type II restriction enzymes share little sequence homology, they all share a core structure that consists of a five-stranded mixed β-sheet flanked by α-helices. Further, all type II restriction endonucleases have a conserved PD . . . D/EXK catalytic motif, this consensus represents the amino acids proline (P), aspartic acid (D), glutamic acid (E), lysine (K) and where X is any amino acid and where the two carboxylates D and E are responsible for Mg²⁺ binding, essential for cleavage but not for binding to the substrate. While this consensus is conserved throughout all the type II restriction enzymes thus far characterized it is quite relaxed making it difficult to locate the motif by sequence inspection (Pignoud and Jeltsch (2001) NAR, 29(18) 3705-3727). On the other hand, cut-deficient enzymes may be of unanticipated character and can be selected for as has been previously shown (Dorner and Schildkraut (1994) NAR. 25;22(6): 1068-74).

Cut-Deficient Endonucleases.

Recently, methods have been used to mutate and screen various restriction endonucleases to alter their binding specificities and cleavage efficiencies (Xu, S. and Schildkraut, I., Isolation of BamHI Variants with Reduced Cleavage Activities (1991) JBC, 266(7) 4425-4429). Several classes of mutants were isolated including those that have not lost their binding specificities but no longer cut DNA. One such mutant, BamHI D94N (Asp94 to Asn), contains a single amino acid substitution within the binding motif that eliminates its ability to cut DNA. This mutation also increased the equilibrium association constant (K_{A}) to greater than 1,100 fold that of wild-type BamHI. The increase in equilibrium constant essentially allows high affinity binding of the cdRE to its recognition sequence. Other such mutant endonucleases have already been isolated, including BbvCI, BsoBI, BglII, PvuII, EcoRI, EcoRV and NotI.

The high affinity binding of the cdRE for its target sequence provides an excellent way to site-specifically label nucleic acids. In addition, because the cdRE's are expression products, not products of chemical synthesis, they can be mutated and expressed in bacterial systems. FIG. 2 shows an SDS-PAGE gel illustrating progressive purification of the recombinant mutein with 20 mM imidazole washes and elution with 200 mM imidazole.

cdREs and Quantum Dots: Implications for Gene Mapping

The most desirable physical mapping system is one that can easily integrate multiple pieces of information including (1) physical maps from multiple cdREs (or other sequence-specific recognition molecules) (2) the size and linear order of fragments, and (3) additional sequence information of BAC inserts. To keep pace with an ever-increasing demand for genome projects, high-throughput processing is also an essential component of any viable mapping system. The “optical fingerprinting” method, described herein, combines the advantages of traditional fingerprinting and optical mapping, but has the distinct advantage of being able to visualize multiple cdREs of the same or different specificity bound to DNA. Thus, rather than using a single RE that cuts DNA, optical fingerprinting records the linear order of differently colored quantum dots each linked to cdREs of particular specificity on an unbroken DNA molecule. Individual cdREs can be visualized because they are labeled with highly luminescent QDs. Although no fragments are produced per se, the position and length between each adjacent cdRE site is available. Confident assembly of individual contigs is significantly enhanced by analyzing many different cdREs simultaneously, without costly need to integrate data from independent physical maps.

While the following examples describe the use of the invention in conjunction with cut-deficient endonucleases, it should be appreciated that the invention is in no way limited to cut-deficient endonucleases in general or Type II restriction enzymes in particular. Rather, molecules such as cut-deficient endonucleases represent a type of site-specific nucleic acid binding molecule that is further exemplified by various transcription factors, positive or negative (e.g. repressor factors), such as helix-turn-helix proteins, zinc finger proteins, leucine zipper proteins, helix-loop-helix proteins, as well as, nucleic acid specific antibodies, TATA box binding proteins and the like. Further, each of these binding partners has specific binding sites and motifs which allows their use either in combination with other binding partners or alone. It should be appreciated that, the information provided by using the invention with multiple binding partners will differ as the binding partners differ. Thus, for example, structural information and assembly of contigs may be facilitated by the use of one or more cdREs while qualitative information may be obtained by the use of binding partners such as transcription factors including repressor and activator factors and steroid receptors to probe nucleic acids for various regulatory elements within the library inserts.

Various exemplary embodiments of compounds and methods according to this invention will be understood more readily by reference to the following examples, which are provided by way of illustration and are not intended to be limiting in any fashion.

### EXAMPLES

Experimental Design and Methods

Bacteriophage λ provides an excellent model for initial experiments with optical fingerprinting. The chromosome of mature λ is linear double-stranded DNA and 48,502 base pairs in length. λ phages relatively small size and rich distribution of restriction sites for which cdREs are available provides an ideal format in which to test optical fingerprinting methods. Optimization of optical fingerprinting of the bacteriophage λ model also allows its adaptation for mapping BAC clones.

Immobilization of λ DNA

The precise mechanism by which DNA molecules interact with derivatized surfaces (e.g. glass substrates) remains unknown. However, efficient binding and elongation of large DNA molecules to such surfaces requires a balance between fluid flow and electrostatic forces. For example, understretched DNA can remain coiled and occlude restriction sites. Optical fingerprinting, will require linear (uncoiled) DNA so that each potential restriction site is available and unobstructed from overlapping strands of DNA. Moreover, strictly linear DNA will enhance the accuracy of length measurements based on integrated fluorescence intensity (see below). Techniques of DNA immobilization on derivatized surfaces is an active area of research. Highly detailed procedures already exist for many lengths of DNA including bacteriophage λ and BAC clones. However, there are advantages of performing the binding step in solution and then immobilizing the complex on a derivatized surface. For example, although the mechanism remains undetermined, the substantial rates of false positives and negatives in traditional optical mapping are likely due to interaction of the nucleic acid with the derivatized mounting surface. Steric hindrances prevent the binding partner from binding and/or cutting the DNA. Such hindrances are not present in solution where a polynucleotide is free to flex and its surface residues are available to the solvent, much as is true in its native, biological, environment. In a preferred embodiment, a polynucleotide is first labeled with one or more site-specific binding partners in solution and then mounted on a derivatized surface.

Cut Deficient BamHI-Labeled with Green Quantum Dots

Initial investigations demonstrate that QD-labeled cdREs can stably bind to immobilized λ DNA and be visualized with confocal microscopy. First, D94N was labeled with green QDs and delivered to full-length λ chromosomes immobilized on a glass substrate. Double-stranded DNA was then stained with an intercalating dye with an emission spectra distinct from the QDs (e.g. YOYO-1; Molecular Bioprobes, Invitrogen, Carlsbad, Calif.). These molecules are easily visualized using commercially available microscopy, such as, for example, a TCS SP2 AOBS confocal microscope (Leica, Mannheim, Del.). The λ chromosome has 5 BamHI sites located at nucleotide positions 5505, 22346, 27972, 34499, and 41732. The Leica system exhibits a physical resolution of 170 nm that corresponds to approximately 500 bp of linear DNA. Adjacent BamHI sites are separated by 16,841, 5,626, 6,527 and 7,233 bp. Therefore, it is expected that 5 distinct sources of QD-generated light corresponding to bound D94N protein will be observed.

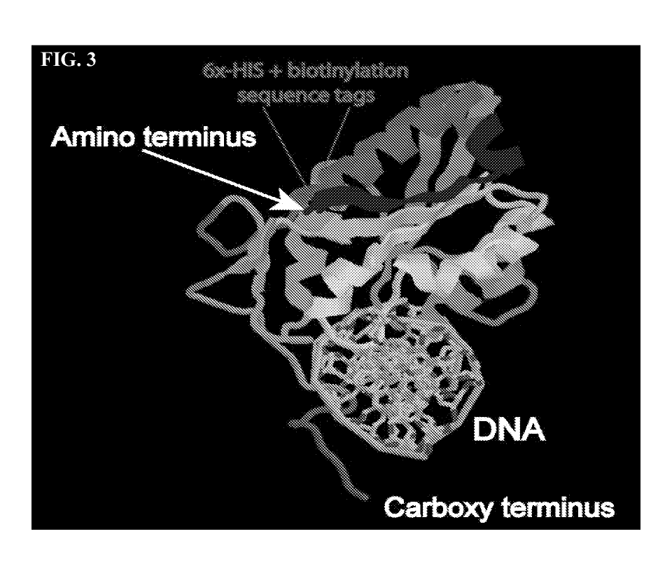

The crystal structure of BamHI (FIG. 3) indicates that 30 surface amino acid residues containing primary amines are available for bioconjugation (25 Lys; 5 Arg). It has previously been reported that PLM solubilized QDs contain only a single QD, a number sufficient to visualize with light microscopy. However, the inventors have found that many of the bioconjugatable surface amino acids are in or near the binding cleft of D94N. Moreover, the inventors have observed that non-specific labeling of D94N with QDs appears to often occlude the binding site. Therefore, the inventors indirectly label labeled the complex using a recombinantly engineered biotin conjugated D94N followed by labeling with a QD linked streptavidin, commonly used in QD imaging studies of chromosomes and actin filaments and commercially available from, for example Quantum Dot Corporation (Hayward, Calif.). These studies reported that signals from QD-conjugated streptavidin were significantly brighter than organic dyes. The conjugates did not exhibit any reduction in spectral intensity even under conditions where rhodamine conjugates experienced a total loss of photon emission. Although the precise location of bioconjugatable sites on streptavidin has not been performed with QDS, there is no evidence that any of the binding sites are occluded. Advantageously, the timescale of spectral collection can be adjusted to a scale of minutes and is more than sufficient for visualization without loss of spectral signal or interference from the blinking phenomenon.

Cut Deficient BglII-Label with Orange Quantum Dot

The ability to multiplex different QD-labeled cdREs and simultaneously detect their linear order on an unbroken DNA molecule is a unique feature of optical fingerprinting. To accomplish this, the limits of resolution between adjacent cdREs must be determined. In the same manner as D94N, a second cdRE, BglII, can be labeled with orange quantum dots and delivered to full-length λ chromosomes immobilized on a glass substrate. The λ chromosome has 6 BglII sites at nucleotide positions 415, 22425, 35711, 38103, 38754, and 38814. In contrast to BamHI sites, distances between adjacent BglII sites are incrementally closer to each other: 22,010, 13,286, 2,392, 651, and 60 bp. The first 3 sites should appear as discrete point sources of light. However, that discrimination will become more difficult between sites 38103 and 38754 as a distance of 651 bp is close to the physical resolution limit of the microscope (500 bp). It may be difficult to resolve the last two sites as they are separated by only 60 bp. Nevertheless, the magnitude of resolution afforded by an average resolution of even 1 Kb is still an order of magnitude greater than that achieved in the best optical maps currently available.

Even if new technologies overcome the limits of current optical microscopy, steric hindrances of cdRE protein binding may pose a problem in discriminating between recognition sites that are extremely close to each other. If an average globular protein is approximately 3 nm in diameter, it would occupy approximately 10 bp when physically bound to DNA. The conjugation of QDs ranging in size from 4-12 nm (green though orange) onto the protein's surface increases the range of coverage to at least 33 bp and up to 80 bp, respectively. Although physically determining the binding events to closely proximal sites, using BglII alone and BglII with BamHI, may be difficult, this issue is addressed by observing the spectral characteristics of closely adjacent cdRE sites. The difference of 60 bp between BglII sites 38754 and 38814 provides an ideal test case as we the range of DNA coverage with orange QDs is estimated to be less than 40 bp. If no steric hindrance exists, two bound BglII cdREs should exhibit twice the spectral intensity of a single BglII cdRE. Visualization with BamHI will provide additional clarification. For example, the length of DNA between BamHI site 22346 and BglII site 22425 is 79 bp. If spectral emissions from both QDs are detected at that site, then it would be concluded that both cdREs are present and not affected by steric interactions. However, if spectral emissions of only a single color are observed at that site but they vary in color between independent λ chromosomes, it must be concluded that steric hindrance exists and there is a competition for the binding site between the two different cdREs.

Visualization with Confocal or Wide-field Microscopy

The TCS SP2 AOBS filter-free spectral confocal and multiphoton microscope is equipped with 4 lasers capable of simultaneous spectral imaging. The 405 nm diode laser and the 594 HeNe laser can be used to excite the QDs and YOYO labels, respectively. Compared to other confocal microscopes, the AOBS (acousto-optical beam splitter) system eliminates conventional dichroic mirrors composed of glass. This feature substantially increases the optical sensitivity of the instrument. The microscope is equipped with a PMT-based detector capable of complete collection of spectral emissions at a resolution of 4096×4096 pixels on a 12-bit intensity scale. New advances in nanoscopy and commercialization of these products are rapidly improving optical resolution well beyond the previous limits imposed by diffraction. A 4Pi system capable of 100 nm lateral resolution is also commercially available. New technologies have proven that resolutions of 80 nm and up to 28 nm can readily be achieved.

Development of Fiducial Methods for BAC Clones

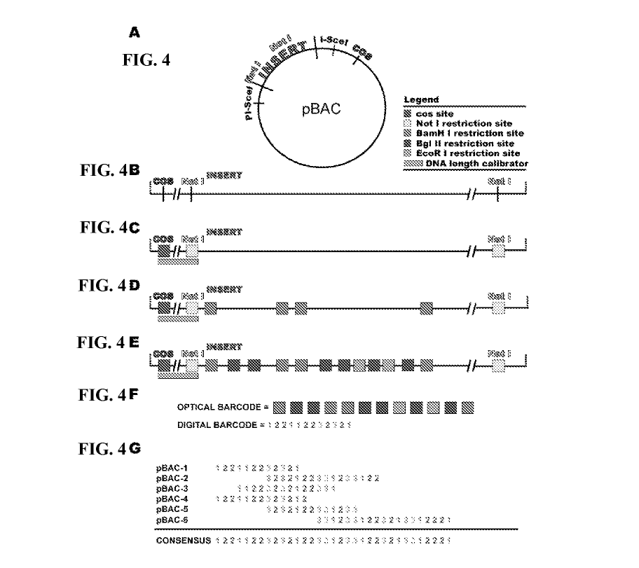

Bacteriophage λ methods will be adapted for optical fingerprinting technology for use in BAC clones (FIG. 4A). Four steps are involved.

Insert Start- and End-Points

The ability to determine insert sizes provides important information concerning the quality of the overall library, aids the construction of contigs, and ultimately the entire physical map. Flanking NotI sites in pBAC clones can be used to define the extent of the BAC insert (FIG. 4B). A Not I cdRE is commercially available (New England Biolabs, Ipswich, Mass.) and can be used to label the terminal ends of the insert with QDs of an emission spectrum distinguishable from the other cdREs. Even when NotI sites are present in the insert, the ability to orient the BAC (below) will still allow identification of the terminal ends of the insert. Even when the insert is retained within the vector.

Orientation of BAC DNA

During immobilization, endpoints of the linear DNA molecules are distributed in random directions. A reference point, common to all BAC clones, is used as a standard by which to gauge lengths of DNA (below). The small subunit of bacteriophage λ terminase, gpNu1 recognizes the cosB subsite of cos. Cos is adjacent to the I-Scel site used to linearize the pBAC clones and I-Scel is adjacent to the insert start site. Thus, determination of the Cos site identifies the insert start site for each insert randomly immobilized on the substrate. Additionally, by labeling each gpNu1 with a larger diameter QD, emitting at a longer wavelength of ˜650 nm, in the red spectrum, smaller-diameter QDs, emitting at wavelengths roughly between 500 and 600 nm, are conserved for labeling of internal sites on the insert (FIG. 4C).

Use of a DNA Length Calibrator

The amount of information contained in the linear sequence of cdREs is a significant improvement over existing methods. However, it is desirable to know the distance, in bp, between specific cdRE binding sites to facilitate the construction and verification of contig assemblies. A length standard is calculated using estimates based on the length of DNA between the cos region and the proximal NotI site. To obtain this estimate, the spectral intensity of DNA stained with YOYO-1 between the two QD-features is measured (FIGS. 4C, 4D, and 4E). The brightness of the DNA “fragment” is proportional to the number of intercalated YOYO-1 molecules and therefore proportional to the DNA fragment length. Others have shown that calibration estimates improve with increasing lengths of DNA. Fragments longer than 4 kb exhibited errors less than 10 percent, which is comparable to the performance of gel electrophoresis. The use of elongated linear DNA improves these estimates significantly.

Assembly of Contigs

The final step in BAC clone analysis is the integration of individual contigs into an ordered, contiguous map. Optical fingerprinting identifies a linear series of specific sites derived from many different cdREs (FIG. 4E, 4F). For example, a 150 kb BAC insert will contain 36 recognition sites, on average, for a 6-bp RE. Fingerprinting with 4 cdREs increases the number of sites to 144, on average. Compared to traditional optical mapping, the amount of information contained in the fingerprint is significantly greater. Contig assembly with fingerprints is less complicated than that required for optical map construction. For example, when 4 cdREs are used to generate a fingerprint, contig construction is derived directly from well-known rules already implemented in DNA contig assembly algorithms (FIG. 4G). This approach takes advantage of the ability of cdREs to saturate all available binding sites (i.e. produce homogeneous fingerprints), in part, because of their higher binding affinity, compared to REs which have only an approximately 60 percent digestion efficiency when digesting immobilized DNA. This inefficiency requires the construction of hypothetical maps from a compilation of incomplete cleavage maps requiring sophisticated algorithms to construct.

Visualization with High-Throughput Methods

The optical fingerprinting system is adaptable to high-throughput platforms capable of processing thousands of BAC clones in a reasonable amount of time. This is achieved through the use of nanochannel arrays and microfluidics as tools to parallelize the detection of optical fingerprints. For example, a channel 10 mm long, 500 nm wide and 500 nm deep provides space for thousands of elongated linear DNA molecules. Even if each channel was confined to a lateral space of 20 μm to accommodate the required microfluidics, there still is room for 500 channels in a 1 cm×1 cm array. Such a device processes BAC clones organized in 96- or 384-well plates. This technology may also overcome problems associated with conformational occlusion of binding sites, should it occur. In that instance, the binding of cdREs could first take place in free solution. The molecules of DNA could then be passed through a nanochannel array where excitation of QDs occurs, and a recording device collects the spectral information in the form of an optical fingerprint.

### Example 1

### Purification of Recombinant Cut-Deficient BamHI

A histidine tag was fused to the N-terminal end of the D94N sequence and it was ligated into the T7 expression vector pIVEX (Roche Diagnostics, Indianapolis, Ind.). This construct was electroporated into a MDS42 E. coli strain (Scarab Genomics, Madison, Wis.) containing T7 polymerase regulated by the auto-inducible rhamnose operon. This procedure was required as it was discovered that even tiny amounts of expression (e.g. from a “leaky” promoter) prior to induction in a standard host cell (such as, for example JM109) failed to produce measurable amounts of D94N and even induced numerous mutations in the D94N sequence. This problem was solved using MDS42 cells as an expression host. Ni-NTA purification of the expression product typically yields more than 100 mg protein per 250 ml culture. This product is easily purified and washed using imidazole as shown in FIG. 2.

### Example 2

### Bioconjugation of QDs to cdRE

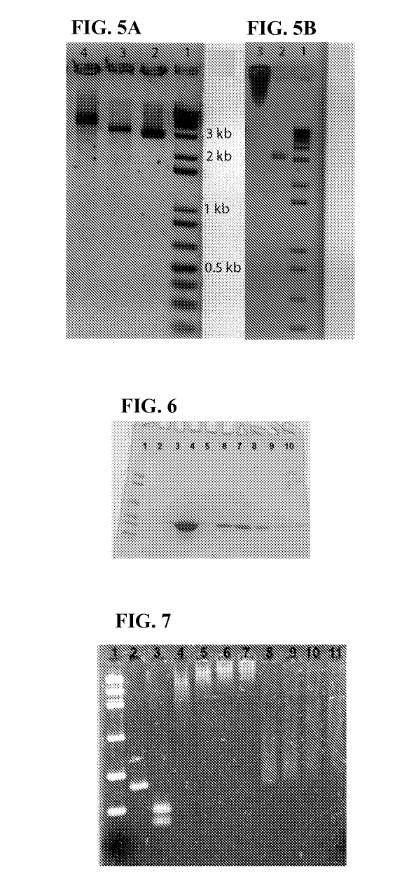

Recombinant D94N molecules were used in an electrophoretic mobility shift assay using 1.5% agarose. FIGS. 5A and 5B show the results of mobility shift assays with the D94N protein. FIG. 5A EtBr stained gel of BpmI digested pGEM3Z with unlabelled D94N: Lane 1, marker; lane 2, pGEM3Z; lane 3, pGEM3Z+D94N cell lysate; lane 4, pGEM3Z+purified 6×His-D94N. FIG. 5B QD-conjugated D94N: lane 1, marker; lane 2, pGEM3Z; lane 3, pGEM3Z+QD labeled 6×His-D94N. The QD-labeled D94N proteins caused an enhanced mobility shift of BpmI-linearized pGEM-3Z (compare FIG. 5B, lane 3 with FIG. 5A, lane 4). The results demonstrate that D94N binds stably to the BamHI site in pGEM-3Z (FIG. 5A), but does not cleave the DNA. This shows that the D94N protein, even with the HIS tag attached, behaves as the native D94N. Similar mobility shifts are observed with D94N/QD bioconjugates where the QDs have surface-capping layers consisting of DTT, cysteine, or mercaptoacetic acid (not shown). In the image shown in FIGS. 5A and 5B it is not possible to distinguish between the QD-labeled D94N signal and the EtBr stained DNA. In contrast to the shifts induced by D94N alone (FIG. 5A), the QD-bioconjugate exhibited a smear rather than a discrete band of shifted DNA (FIG. 5B). Although no firm conclusions can be made, the smear may indicate the presence of unbound QD-bioconjugate either due to simple excess or because of binding-site occlusion. The crystal structure of BamHI (FIG. 3) indicates a large density of primary amine-containing amino acids (lysine) located in or near the DNA binding site. As 1-ethyl-3-(3-(dimethylamino)propyl)carbodiimide hydrochloride (EDC)-mediated bioconjugation of carboxylated QDs to primary amines is non-specific, some or many QD-bioconjugates are likely to have attached at the DNA binding site. This result emphasizes the value of indirect labeling methods such as with biotin/streptavidin, antigen/antibody or the like.

### Example 3

### Quantum Dot Labeling of Cut-Deficient Restriction Enzymes: Site-Specific Biotinylation of D94N

To correct the problem of binding-site occlusion a new D94N protein containing a biotinylation tag, a 15 amino acid sequence that is specifically biotinylated by the E. coli birA gene was constructed. The N-terminal placement of the tag is almost completely opposite to the DNA binding site (FIG. 3) and is freely available for interaction with streptavidin, to which QDs directly can be directly bioconjugated.

In vivo biotinylation may be more efficient than in vitro methods. Therefore, the D94N gene containing the biotin target peptide sequence and the E. coli biotinylation gene birA were co-transformed into MDS42. In this investigation the inventors chose to put the D94N and birA constructs are on separate T7-promoter vectors containing ampicillin and chloramphenicol markers respectively. The unmodified birA gene was placed into a CAM-selectable single-copy vector (pKG15) under pBAD (Invitrogen, Carlsbad, Calif.) control. Auto-inducible expression occurred in standard translation buffer (TB) at 30 C for 16 hrs. Biotinylated D94N was isolated and purified in two distinct steps. First, Ni-NTA columns isolated D94N from the crude cell lysate using the 6×-histidine tag still present on the N-terminus of the D94N biotinylation sequence-tagged construct. Second, monomeric avidin column (Pierce Scientific, Rockford, Ill.) was used to separate the biotinylated D94N (b-D94N) from the un-biotinylated D94N protein. The immobilized D94N was eluted from the avidin column with a suitable buffer containing 1 mM d-biotin. Excess d-biotin was removed by passing the D94N through a Sephadex G-25 column. The amount of purified b-D94N recovered (FIG. 6, lanes 6-10) was nearly all of the original protein obtained from the Ni-NTA purification. The amounts of b-D94N in lanes 6-10 are not indicative of biotinylation efficiency of D94N per se, rather, analysis indicates that the avidin column is saturated from the initial application of Ni-NTA purified b-D94N protein. Repeatedly passing the flow-through of the original sample (FIG. 6, lane 4) over recharged monomeric avidin columns results in recovery of almost the same amount of initial D94N protein, indicating that in vivo biotinylation of tagged D94N is highly efficient in the described system.

Gel shift assays of purified b-D94N (FIG. 7) show no difference in site-specific binding ability in comparison to un-tagged D94N (FIG. 2). Although QDs are bioconjugated to streptavidin molecules non-specifically, there is substantial evidence that none of the four binding sites are occluded by QDs. This result demonstrates that conjugation of PLM-coated QDs do not appear to interfere with the binding of D94N to DNA.

### Example 4

### Visualization with Confocal Microscopy

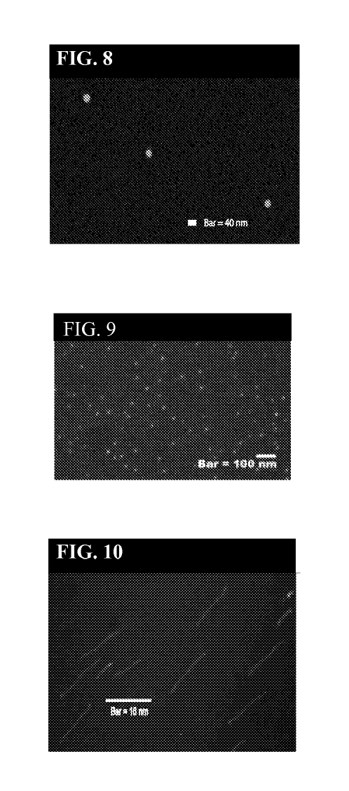

Phospholipidmicelle (PLM)-coated QDs were first bioconjugated to the D94N protein using standard EDC-based condensation chemistries and visualized using a TCS SP2 AOBS confocal microscope (Leica). The intense fluorescence of the QD-labeled D94N protein is clearly visible from slight background scatter (FIG. 8). The diameter of the D94N/QD bioconjugate was estimated to be approximately 32 nm (scale bar=40 nm). Given that the diameter of the D94N protein is approximately 10 nm, at least two QDs (estimated diameter=10 nm) must be attached at positions nearly opposite to each other. D94N protein labeled with green QDs (capping-layer=mercaptoacetic acid) was visualized with the TCS SP2 AOBS confocal microscope with 405 nm excitation.

### Example 5

### QD-Labeled Streptavidin Binds Biotinylated D94N

Multimeric streptavidin was purchased commercially and bioconjugated to silane-protected QDs with standard EDC chemistry. Although QDs are bioconjugated to streptavidin molecules non-specifically, there is substantial evidence that none of the four binding sites are occluded by QDs and excellent recovery of QD-labeled streptavidin bound to b-D94N obtained was obtained (FIG. 9).

### Example 6

### Visualization of Immobilized DNA on a Glass Substrate

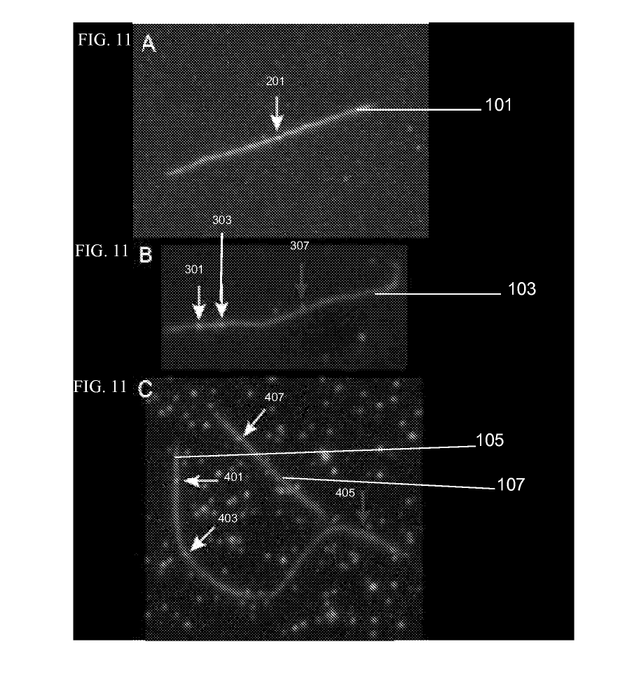

Optically-flat glass slides were cleaned with an acid wash (3:1 HNO₃ and HCl) and modified with amino-propyl tri-ethoxysilane (APTES) by vapor deposition. Approximately 1 ng of phage λ DNA intercalated with YOYO-1 dye was applied to the APTES-coated surface. The DNA molecules (polynucleotides) were visualized with the Leica confocal microscope using 488 nm illumination. FIG. 10 shows that the method straightens full-length λ DNA molecules. Lambda DNA (48.5 kb) has a theoretical length of approximately 16.5 um which is very close to our observed length of 17-18 um. Differences in the estimated overall length of DNA result from the degree of stretching, a function of the local surface-charge density. The vapor-deposition method has provided more consistent results than obtained with other methods.

### Example 7

### BamHI Bound λ DNA

The sequence specific binding of a cdRE to a polynucleotide was studied using b-D94N and λ DNA. FIGS. 11A, 11B and 11C show optical fingerprinting in which quantum dots are used to visualize the position of BamHI specific sequences on DNA. The His tag and a birA biotinylation peptide (EXAMPLE 2) b-D94N was bound to immobilized λ DNA 101 and visualized with a QD-streptavidin bioconjugate 201 (FIG. 11A). This was accomplished by first elongating YOYO-1 stained λ DNA 101 (green) on 3-aminopropyltriethoxysilane (APTES) derivatized glass slides with QD-labeled streptavidin (red) 201 shown in FIG. 11A. Five BamHI sites were expected to be illuminated with a single QD, however, the observed λ molecules labeled typically with only one or two QDs shown in FIG. 11B. Initial criteria for determining a binding event are: (1) a color-shift of the red QD to yellow. When red and green fluorescence is combined, yellow is the resulting color. The degree of color-shift is determined by the orientation of the QD relative to the DNA; (2) the position of the QD must be radially oriented and highly proximal with respect to the DNA. When a bound QD is juxtaposed with DNA, it often appears as a “bump” on the DNA with a slight yellow color shift at the DNA:QD interface. This phenomenon likely results from the distance added by the b-D94N, streptavidin, and QD (ca. 10 nm; the axial width of DNA is about 4 nm). If the QD is directly above the DNA, the complex appears to be in line with the DNA and exhibits a striking yellow color.

To determine the position of the bound b-D94N, the spectral intensity of YOYO-1 was integrated along unlabeled, stretched DNA molecules and scaled in intensity to the length of λ (48,502 nt). YOYO-1 intensity was measured from the ends of the candidate molecule to the position of the QD. The distance in nucleotides was determined by linear regression against the standard molecules. Calculations of the known and observed BamHI positions showed good agreement (301 and 303 FIG. 11B and 401, 403 and 407 11C). To address issue of incomplete labeling, a similar experiment was performed but with active BamHI endonuclease for b-D94N. Since λ DNA molecule is under slight tension, cuts appear as distinct breaks in an otherwise contiguous DNA molecule. Surprisingly, the same pattern was observed with active BamHI as with b-D94N: many λ molecules were uncut, and those that were usually exhibited only one or two cuts. This result suggests strongly that the DNA may be adhered too tightly to the derivatized slide surface thereby occluding the clefted binding site of b-D94N/BamHI. This issue can be addressed by (1) reducing the density of APTES derivatization; and (2) performing b-D94N binding in solution. A combination of both approaches may also be used to take full advantage of the multiplexing capability it offers.

### Example 8

### DNA Microarray

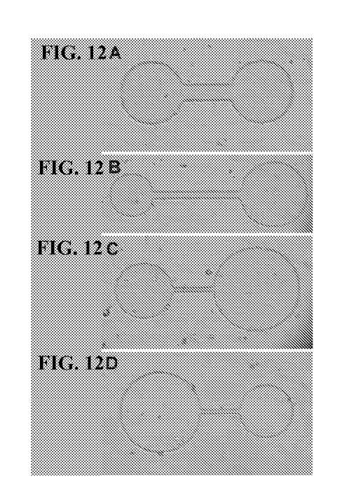

In order to scale up the optical fingerprinting process to view up to 1000 different BACs per slide, a prototype array was designed to elongate DNA molecules in a microarray format. The basic design (FIGS. 12A-12D) consists of an input well for fluid deposition (left), an elongation channel where the DNA stretches and adheres to a derivatized substrate (middle), and an output well (right) for fluid and pressure release. Two blocking masks were manufactured to control the deposition of polydimethylsiloxane (PDMS) polymer. The bottom mask formed the elongation channels while the top mask formed the input and output wells. The first prototype array fit onto a 1″×3″ glass slide and contained 200 variations on the basic design including: channel length; channel width; input well diameter; and output well diameter. Fluid flow across the channels occurs spontaneously when the PDMS array is seated on glass slides derivatized with APTES (3-aminopropyltriethoxysilane), a well-characterized DNA binding silane. Optimization of these parameters can be used to determine specific design and buffer conditions that will best elongate and stabilize phage λ DNA molecules and can be used in automated fluid-loading systems capable of accommodating the expected unit density (>7000 per slide) for high-throughput analysis.

### Example 9

### Use of D94N with Biotinylated Secondary Antibody

Previous experiments performed by the inventors utilized a cutting deficient restriction enzyme conjugated to a reporter molecule via a covalently attached biotin moiety. However, the use of cut deficient enzymes need not be limited to those that were directly labeled with a reporter molecule. Therefore, the inventors prepared a further series of experiments to show that the D94N cdRe can be prepared recombinantly with only a 6×HIS tag and labeled indirectly with a commercially available anti-6×HIS antibody that is also site-specifically biotinylated. Using this method, the cdRE bearing the 6×HIS tag is contacted with a biotinylated anti-6×HIS antibody which is then available for labeling with QD-streptavidin biocongugates as described previously. This style of labeling is not limited only to the interaction of biotin and streptavidin. Any of a variety of QD-labeled antibodies specific to the anti-6×HIS antibody would also serve to indirectly label the cdRE. Moreover, the specific antigen recognized by the antibody need not be only 6×HIS tag. Any such antigen capable of being integrated into the cdRE may serve as a recognition moiety. Such antibodies are commercially available from, for example, Qiagen (Valencia, Calif.), GE Healthcare (Milwaukee, Wis.). For purposes of experimentation and demonstration, another cdRE was engineered, D94N-11, which only contains a 6×HIS tag, and in contrast to D94N-32 which contains both 6×HIS and an AVITAG.

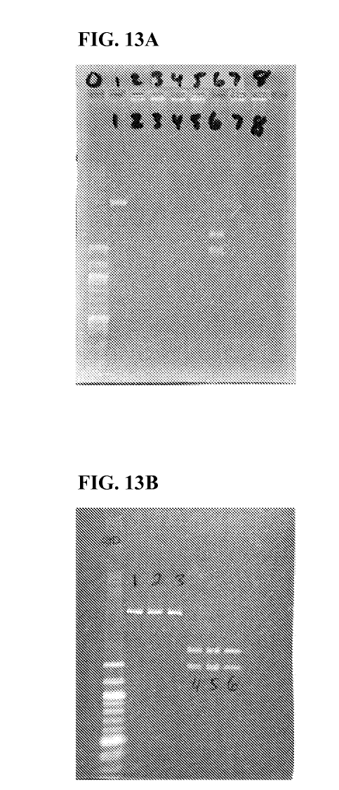

With regard to this embodiment, FIG. 13A is an image of a polyacrylamide gel showing the effects of a gel shift where: Lane 0: Molecular weight standard; Lane 1: ScaI cut pGEM3Z; Lane 2: D94N-11+pGEM3Z (gel shift with cdRE D94N-11 with 6×HIS tag only); Lane 3: D94N-32+pGEM3Z (gel shift with cdRE D94N-32 with 6×HIS tag and AVITAG (AVIDITY, Denver, Colo.); Note: mixture of biotinylated/un-biotinylated forms); Lane 4: As lane 2 but with active BamHI added; Lane 5: As lane 3 but with active BamHI added; Lane 6: As lane 1 but with active BamHI added (positive cut control of single BamHI site in pGEM3Z); Lane 7: Replicate of lane 4; Lane 8: Replicate of lane 5. The results of this experiment show that D94N-11 and D94N-32 bind to the BamHI site in ScaI cut pGEM3Z [lanes 2, 3] and protect it from cleavage from active BamHI [lanes 4, 5, 7, 8]. The 6×HIS and AVITAGs either alone or combined do not adversely affect the site-specific binding ability of D94N.

FIG. 13B is an image of a polyacrylamide gel showing the effects of a gel shift where: Lane 0: Molecular weight standard; Lane 1: ScaI cut pGEM3Z; Lane 2: purified biotinylated D94N-32 (b-D94N-32) Note: no longer a mixture of biotinylated and un-biotinylated forms; Lane 3: replicate of lane 2; Lane 4: Lane 2+active BamHI; Lane 5: replicate of lane 4; Lane 6: As lane 1 but with active BamHI added (positive cut control of single BamHI site in pGEM3Z). The results of this experiment show that, when purified from unbiotinylated D94N-32, b-D94N-32 biotinylated D94N-32 (b-D94N-32) is less able to protect active BamHI from cleaving the BamHI site in ScaI cut pGEM3Z [lanes 4, 5]. Since un-biotinylated D94N-32 is able to protect pGEM3Z from cleavage, the addition of the biotin moiety somewhat affects the DNA binding function of the protein. The gel shift assay is not sensitive enough to determine the reduction in binding efficiency. However, the inventors have evidence that b-D94N-32 (biotinylated D94N) is certainly not eliminated completely having obtained images of b-D94N-32 bound to DNA which have been conjugated to a Qdot-streptavidin reporter (See, FIG. 11). These data indicate that both strategies are viable for identifying or labeling the location of the cdRE. While the current location of the biotin conjugated cdRE may result in greater steric hindrance to the binding of the cdRE to the binding site, the inventors expect that by either changing the linear order of the 6×HIS and AVITAGs or by eliminating the 6×HIS tag through routine experimentation, the binding affinity will be increased. Alternatively, the use of biotinylated anti-6×HIS antibodies can be used to visualize D94N-11. Further, those of skill in the art will recognize that secondary antibodies having reporter moieties including Q-dots, or fluorescent moieties such as are commercially available can also be utilized to visualize the anti-6HIS antibody (see, for example, tebu-bio (Peterborough, UK) and Invitrogen (Carlsbad, Calif.)).

It should be apparent to those of skill in the art that the invention described herein can be used with any site-specific nucleic acid binding partner. Binding of each partner will impart sequence-specific identification of each polynucleotide regardless of whether the binding partner recognizes a restriction/methylation site or recognizes a functional response element. Moreover, the ability to use both restriction sites and functional sites in the invention provides a powerful high-throughput method to, not only assemble library inserts into contigs, but also to accurately identify the promoter sites of genes and their regulatory elements. Further, it should be appreciated that the invention can be performed in solution and that the ends of the polynucleotide inserts are easily identified regardless of whether the insert is excised from the plasmid. This ability both eliminates the step of excising the insert from the plasmid and also obviates the need to adhere the excised insert to a substrate thereby decreasing the incidence of false positives. Methods of visualizing the placement of the site-specific markers in this invention can include, but are not limited to, the direct conjugation of reporter moieties, such as quantum dots, fluorophors or biotin, directly to the site-specific markers. Alternatively, the site-specific markers can be identified indirectly by, e.g., labeling the site specific marker with an antibody having a reporter moiety attached thereto or a secondary antibody having a reporter moiety attached thereto. For example, as described above, the cdRE may have a 6×HIS tag. This allows the user to probe the tag with an anti-6×HIS antibody that may be directly conjugated to the reporter moiety. Conversely, the anti-6×HIS antibody may be contacted with a secondary antibody having the reporter moiety conjugated thereto. Such immunohistochemical techniques are well known to those of skill in the art.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

What is claimed is:

1. A method of mapping a site of known sequence on a polynucleotide having first and second ends, comprising:
contacting the polynucleotide with a sequence-specific binding partner without scission of the polynucleotide; and
determining the position relative to a first landmark of the sequence-specific binding partner to map the position of a site of known sequence on the polynucleotide, wherein the site of the binding partner is mapped on the polynucleotide.

2. The method of claim 1, wherein the first landmark is selected from the group consisting of: an end of the polynucleotide, an insertion site of the polynucleotide, a cos site and a NotI site.

3. The method of claim 1, wherein the polynucleotide is contained within a vector or plasmid.

4. The method of claim 1, further comprising another landmark that is a sequence-specific binding partner.

5. The method of claim 1, wherein the binding partner comprises a peptide or a peptide nucleic acid.

6. The method of claim 5, wherein the binding partner comprises the DNA binding domain selected from the group consisting of a cut-deficient restriction endonuclease, an RNA polymerase, a steroid receptor and a transcription factor.

7. The method of claim 6, wherein the cut deficient endonuclease is a mutein of an endonuclease having a six residue recognition sequence.

8. The method of claim 6, wherein the transcription factor binding domain is selected from the group consisting of: helix-turn-helix proteins, zinc-finger proteins, leucine zipper proteins and helix-loop-helix proteins.

9. The method of claim 1, wherein the binding partner further is linked to a detectable label.

10. The method of claim 9, wherein the binding partner is directly linked to the detectable label.

11. The method of claim 9, wherein the binding partner is indirectly linked to the detectable label.

12. The method of claim 9, wherein the binding partner is linked to the detectable label by an avidin-biotin or antibody-antigen association.

13. The method of claim 9, wherein the detectable label is a fluorophore, a chromophore, a quantum dot or a radionuclide.

14. The method of claim 1, wherein contacting occurs when the polynucleotide is in solution or adhered to a substrate.

15. A microarray for use in fingerprinting nucleic acids comprising a nucleic acid deposition apparatus, the nucleic acid deposition apparatus including:
a first chamber and a second chamber;
an elongation channel, the elongation channel connecting the first chamber to the second chamber;
a derivatized substrate adhered to the elongation channel;
wherein the first chamber comprises an input well, the second chamber provides an output well and the elongation channel provides a nucleic acid deposition surface.

16. The microarray of claim 15, wherein the first and second chamber are formed by a top blocking mask and wherein the elongation channel is formed from a bottom blocking mask.

17. The microarray of claim 16, wherein the top and bottom blocking masks are formed from polydimethylsiloxane (PDMS).

18. The microarray of claim 16, wherein the top and bottom blocking masks are placed on a surface.

19. The microarray of claim 18, wherein the surface is a glass surface.

20. The microarray of claim 19, wherein the glass surface is derivatized with 3-aminopropyltriethoxysilane (APTES).

21. The microarray of claim 15, wherein at least 5000 nucleic acid deposition apparatus are present on a derivatized surface.

22. The microarray of claim 15, wherein 10,000 nucleic acid deposition apparatus are present on a derivatized surface.

23. The microarray of claim 15, wherein between 5,000 and 10,000 deposition apparatus are accommodated on a derivatized surface.

24. A method of depositing nucleic acid on a derivatized glass slide comprising the use of a microarray according to claim 14 wherein a solution containing a nucleic acid mixture is place in the input well and wherein flow dynamics causes the solution to pass through the elongation channel to the output well and wherein nucleic acids present in the input well deposited on the derivatized surface of the elongation chamber.

25. The method of claim 24, wherein the nucleic acids deposited on the surface of the elongation channel are fingerprinted to make a contig assembly thereby mapping the nucleic acids in the microarray.

26. A kit for use in mapping sites of known sequence on a polynucleotide comprising: at least one binding partner that site-specifically binds the polynucleotide without resulting in scission of the polynucleotide and a detectable label linked thereto.

27. The kit of claim 26, wherein the binding partner is a detectable label.

28. The kit of claim 26, wherein the binding partner is biotinylated.

29. The kit of claim 26, further including a quantum dot conjugated to an avidin moiety.

30. The kit of claim further including a nucleic acid deposition apparatus according to claim 15.
